(19) United States
(12) Patent Application Publication
(10) Pub. No.: US 2005/0137464 A1
(43) Pub. Date: Jun. 23, 2005

(54) Wireless sensor and sensor initialization device and method

(76) Inventor: Frank C. Bomba, Andover, MA (US)

Correspondence Address: HEWLETT PACKARD COMPANY, P O BOX 272400, 3404 E. HARMONY ROAD, INTELLECTUAL PROPERTY ADMINISTRATION, FORT COLLINS, CO, 80527-2400, US

(21) Appl. No.: 10/744,405

(22) Filed: Dec. 23, 2003

(51) Int. Cl. A61B 5/00; A61B 5/02; A61B 5/04; A61B 5/08

(52) U.S. Cl. 600/300; 600/549; 128/903; 600/485; 600/509; 600/544; 600/546; 600/529

(57) ABSTRACT

A sensor initialization device includes a panel with respective indicia for each of plural wireless sensor functions. For each sensor function indicia, there is a respective initialization circuit coupled to the sensor function indicia for electronic communication through the panel to a wireless sensor unit upon selection of the sensor function. The sensor functions can include body temperature, heart rate, blood pressure, respiratory, electrocardiogram, electroencephalogram, electromyography, electrooculogram, and polysomnography. The panel also has respective indicia for each of plural body placement locations. A respective initialization circuit coupled to the body placement location indicia is electronically communicated through the panel to the wireless sensor unit upon selection of the body placement location indicia.

### BACKGROUND OF THE INVENTION

Wireless medial monitoring systems have been proposed in the prior art. One such system can include a sensor, controller, and transceiver electronics all contained within a wireless sensor patch. The wireless sensor patch monitors a predetermined function and transmits data to a receiver. The receiver sends the data to a computer or monitor for viewing.

The sensor within a wireless sensor patch can be either a temperature sensor, a heart rate sensor, a blood pressure sensor, a respiratory sensor, an electrocardiogram sensor, an electroencephalogram sensor, an electromyography sensor an electrooculogram sensor, or a polysomnography sensor. Thus each wireless sensor patch is only able to monitor a single function as defined by its sensor which is designed at the factory (i.e., a factory set function).

### SUMMARY OF THE INVENTION

There is a need for a generic (multifunctional) wireless sensor which can be user programmable for a plurality of biological/physiological properties. In such a case, each sensor would have a unique identifier associated with a specific patient such that a receiving unit is able to determine which sensor is associated with which patient.

The present invention provides a sensor initialization device for such generic (multifunctional) wireless sensors and/or other sensors. The present invention includes (i) a panel with respective indicia for each of plural wireless sensor functions, such that there are a plurality of sensor function indicia, and (ii) for each sensor function indicia, a respective initialization circuit coupled thereto. Each initialization circuit is coupled to the corresponding sensor function indicia for electronic communication through the panel to a wireless sensor unit upon selection of the sensor function. The sensor functions can include body temperature, heart rate, audio (for auscultation, lung sounds, patient vocalizations, etc.), blood pressure, respiratory, electrocardiogram, electroencephalogram, electromyography, electrooculogram, and polysomnography.

There is also provided by the present invention a generic biosensor having (i) a plurality of configurable sensors for sensing biological/physiological functions, (ii) a control unit for programming the biosensor to sense at least one biological/physiological function, and (iii) a transceiver for transmitting the at least one sensed biological/physiological function to a base unit and for receiving programming information from an initialization unit.

A method is provided for initializing a generic biosensor and includes activating the biosensor, programming the biosensor with patient identification information, determining at least one biological/physiological function to be sensed, programming the biosensor for the determined biological/physiological function using an indicia representing the determined biological/physiological function, determining a body location where the biological/physiological sensor is to be placed on the body, and programming the biosensor for the determined body location using an indicia representing the determined body location.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a perspective view of a patient's room using the sensor system of the present invention.

FIG. 2A is a perspective view of a sensor initialization device of the present invention.

FIG. 2B is a circuit diagram for a sensor type programmer of the device of FIG. 2A.

FIG. 2C is a circuit diagram for a sensor body location programmer of the device of FIG. 2A.

FIG. 3A shows an anterior outline of a human body as may be displayed on the panel of the embodiment of FIG. 2A.

FIG. 3B shows a posterior outline of the human body as may be displayed on the panel of the embodiment of FIG. 2A.

FIG. 4 shows a sensor and a kit of sensors employed in one embodiment of the present invention.

FIG. 5 is a circuit diagram of the sensor of FIG. 4.

FIG. 6 is a flow diagram for initializing sensors of FIG. 4 using the initialization device of FIG. 2.

### DETAILED DESCRIPTION OF THE INVENTION

A description of preferred embodiments of the invention follows.

The present invention addresses the need for multi-purpose wireless sensors that are generic across multiple functions (not factory set function wise) which can be programmed or otherwise initialized and activated at the point of use to a specific desired function(s). The same stock or inventory of multi-purpose wireless sensors thus allows monitoring of a plurality of biological/physiological properties. As mentioned above, the present invention accomplishes this by providing a sensor initialization device, a generic biosensor, and a method of initializing generic sensors. With regard to the sensor initialization device, a panel provides a plurality of sensor function indicia and corresponding initialization circuits for electronic communication through the panel. Further, the panel may have respective indicia for each of plural body placement locations, such that there are a plurality of body placement location indicia. For each body placement location indicia, there is a respective initialization circuit coupled to the body placement location indicia. Upon user selection of a body placement location indicia, the corresponding initialization circuit is electronically communicated through the panel to the wireless sensor unit. The body placement location indicia include at least one of a pictorial image of body parts and a pictorial outline of a human or other body with plural user selectable portions. The pictorial outline of the human or other body parts can be segmented into quadrants defining specific regions of the subject body and can include a detailed anterior and posterior outline of the human or other body. The sensor initialization device can also have anterior indicia and posterior indicia for selecting between a side of the human or other body.

The device can include patient identification indicia for programming the sensor with patient identification information. The patient identification indicia can be numerical indicia.

With respect to the invention generic biosensor, each configurable sensor can serve as a temperature sensor, a heart rate sensor, a blood pressure sensor, a respiratory sensor, an electrocardiogram sensor, an electroencephalogram sensor, an electromyography sensor, an electrooculogram sensor, and a polysomnography sensor.

The biosensor can further include unique sensor identification information. The sensor identification information can be a sensor serial number.

The control unit programs the biosensor with patient identification information. The patient identification information can be a patient's social security number, hospital identification number, or any other information which uniquely identifies the patient.

With respect to the invention method of initializing generic sensors, such as biosensors, programming the biosensor with patient identification information includes depressing the biosensor on patient identification indicia representing the patient identification information. The patient identification indicia are any combination of alphabetical, numeral, and other indicia.

Programming the biosensor for the determined biological/physiological function includes depressing the biosensor on biological/physiological function indicia representing the determined biological/physiological function. The biological/physiological function include body temperature, heart rate, blood pressure, respiratory, electrocardiogram, electroencephalogram, electromyography, electrooculogram, and polysomnography.

Programming the biosensor for the determined body location includes depressing the sensor on body location indicia representing the determined body location. Depressing the biosensor on a body location indicia further includes depressing the biosensor on multiple body location indicia until the biosensor is fully programmed with the determined body location. The body location indicia include at least one of a pictorial image of body parts and a pictorial outline of a human or other body with plural user selectable portions. The pictorial outline of the human or other body parts can be segmented into quadrants defining specific regions of the subject body and can include a detailed anterior outline of the human or other body and a detailed posterior outline of the human or other body.

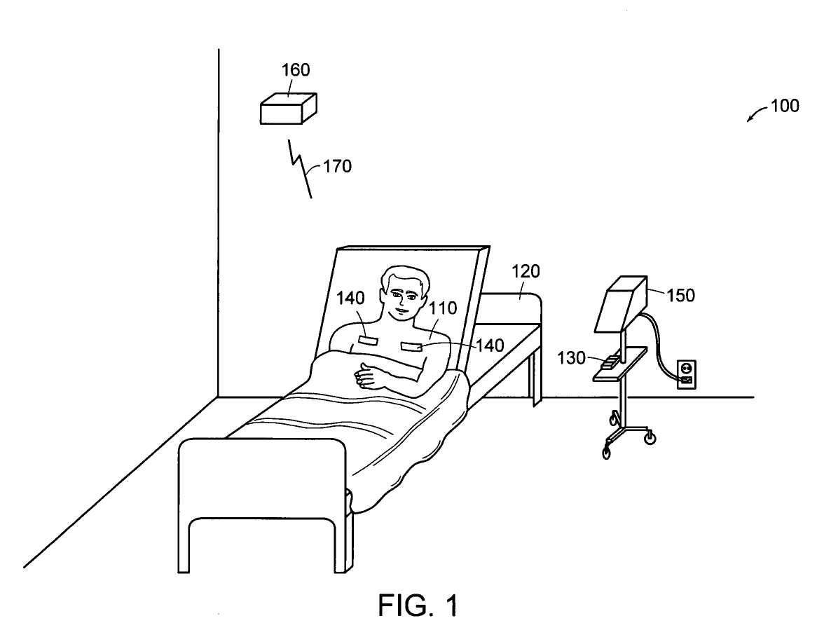

Referring now to the Figs., FIG. 1 shows a sensor system 100 according to the principals of the present invention in use on a patient 110 in a hospital bed 120. In general, a care provider, obtains a generic wireless sensor 140 from a supply (box) 130 of sensors. At this stage the sensor 140 has the ability to monitor any number and variety of functions (biological/physiological properties) and is not a specific/single function sensor as in the prior art. After deciding how he wants to use the sensor 140, the care provider uses initialization unit 150 and initializes the generic wireless sensor 140 for a specific biological/physiological property to be sensed. The care provider than places the initialized sensor 140 on the patient's body 110. The sensor 140 wirelessly transmits data 170 to a base unit 160 which collects information related to the biological/physiological property being sensed. The base unit 160 can be remotely accessed to retrieve the sensed information or the base unit can automatically forward the sensed information to a host computer (not shown). More than one sensor may be used depending upon the physiological property to be sensed.

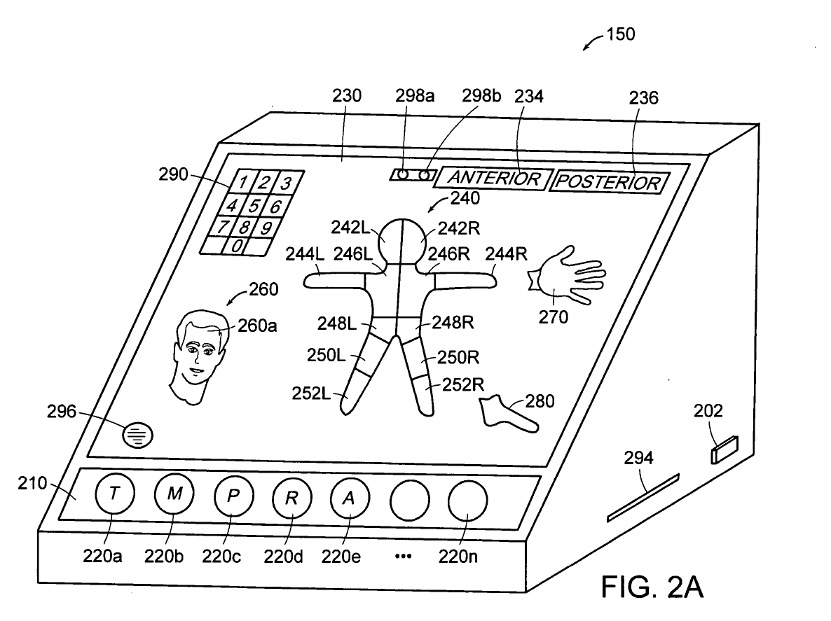

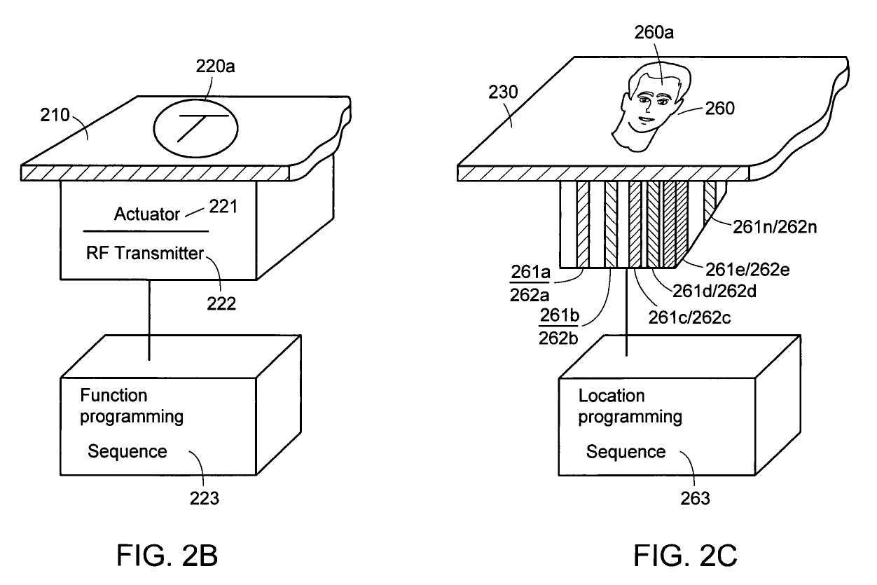

FIG. 2A shows a perspective view of the initialization unit 150. The initialization unit 150 includes a sensor function panel 210, a body location panel 230, and a power switch 202. The sensor function panel 210 includes a plurality of sensor function indicia 220a . . . 220n. Each indicia 220a . . . 220n represents a different type of sensor function. For example, the illustrated indicia “T”, “M”, “P”, “R”, and “A” represent temperature, motion, pulse (heart rate), respiration, and audio respectively. Although “T”, “M”, “P”, “R”, and “A” are shown on the panel 210 of the FIG. 2 embodiment, numerous other types of biological/physiological functions are suitable, such as blood pressure, electrocardiogram, electroencephalogram, electromyography, electrooculogram, and polysomnography. It should be understood that the principals of the present invention apply to any type of biological and physiological functions associated with the human or other body. Behind each sensor function indicia 220a . . . 220n is an associated respective initialization circuit which electronically communicates the sensor function to a wireless sensor 140 (FIG. 1) through the sensor function panel 210.

FIG. 2B shows a sample of an initialization circuit for temperature sensory function corresponding to indicia “T” 220a on sensor function panel 210. An actuator 221 and RF transmitter 222 are located under the panel 210 and in close proximity to indicia “T” 220a. The actuator 221 and RF transmitter 222 are in electrical or optical communication with the sensor function programming sequence 223 associated with indicia “T” 220a. In response to caregiver selection of indicia “T” 220a (e.g. touching of subject sensor 140 to indicia “T”), actuator 221 enables RF transmitter 222 to transmit programming sequence 223 through panel 210 to the wireless sensor 140. Upon receipt of this transmission, wireless sensor 140 processes the received programming sequence 223 which results in initialization (enabling, etc.) of the sensor 140 according to the corresponding function (temperature sensing in this example) of user selected indicia “T” 220a.

Actuator 221 may be pressure induced, heat activated, light sensitive, or of other activation technology. The caregiver may press sensor 140 against indicia of panel 210 to generate corresponding actuators 221, or may otherwise depress desired function panel indicia (to operate corresponding actuators 221) and then hold sensor 140 poised over (near) the indicia for generation of the initialization circuit.

The activation and programming sequence for the sensor function may be, for example in the simple case of applying a thermometer to a patient's forehead. After an initial sequence to input patient ID, the caregiver simply depresses indicia 220a, then 260a before applying to the patient. A more complex sensor application may be required to enable more than one function in the sensor such as may be done to monitor respiration as well as lung sounds by depressing “R” 220d, then “A” 220e in sequence.

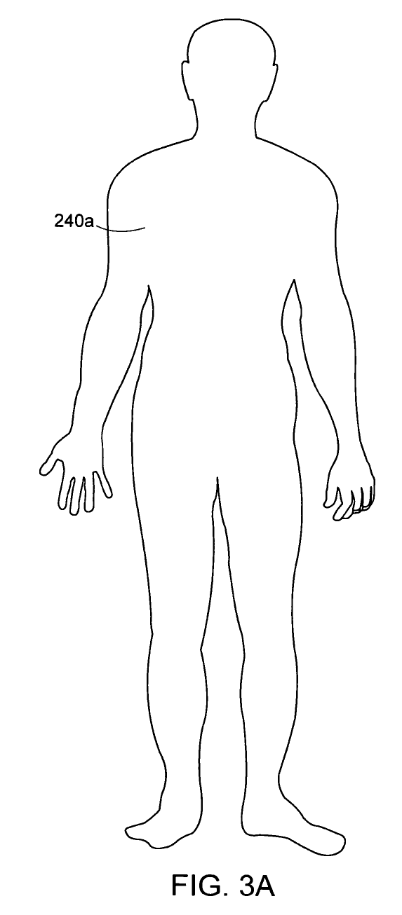

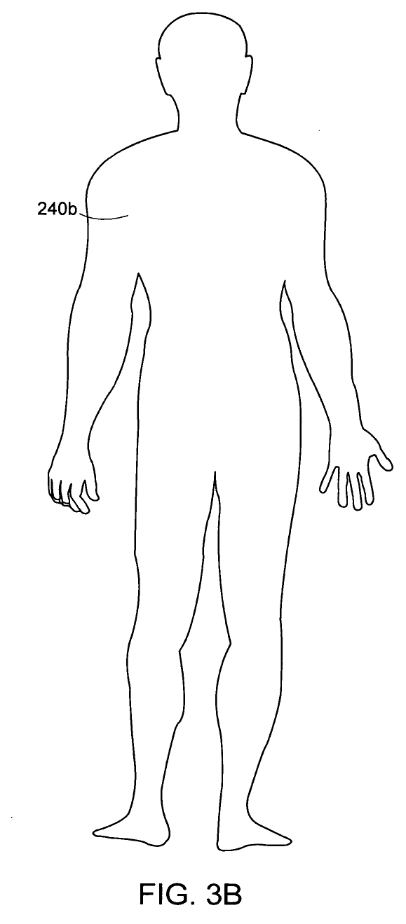

The body placement panel 230 includes a plurality of body placement indicia representing a different location on the human body. These indicia include but are not limited to a human body outline 240, a head 260, a hand 270, and a foot 280. The outline of the human body 240 can be divided into quadrants representing different areas and locations of the human body for a more precise measurement. For example, the body is divided into left and right regions represented by L and R respectively including head region 242, arm regions 244, upper torso region 246, lower torso region 248, upper leg regions 250, and lower leg regions 252. Although 12 quadrants are shown, it should be understood that the quadrant regions can vary depending upon the precision to be obtained. The body placement panel 230 also includes indicia for enabling user selection or specification of the anterior (front) 234 and posterior (back) 236 regions of the body. Upon such user selection, the body outline 240 may be replaced by a detailed representation of the human body's anterior and posterior regions 240a, 240b as shown in FIGS. 3A and 3B. Behind each of the body placement indicia 240, 260, 270, and 280 is an associated respective initialization circuit which electronically communicates the body placement location to a wireless sensor 140 (FIG. 1) through the body placement panel 230.

FIG. 2C is an example initialization circuit for body placement of the forehead 260a for use with the temperature sensing function example of FIG. 2B. A plurality of actuators 261a . . . . 261n and RF transmitters 262a . . . 262n are precisely located under and in close proximity with head indicia 260 representing the precise location the sensor 140 is to be placed on the patient's body, for example the forehead 260a for sensing temperature. The actuators 261a . . . 261n and RF transmitters 262a . . . 262n are in electrical or optical communication with the body location programming sequence 263 associated with head indicia 260. In response to caregiver selection of (e.g. holding sensor 140 against) the forehead area 260a of head indicia 260 on body placement panel 230, corresponding actuator 261 enables its RF transmitter 262 to transmit pertinent location programming sequence 263. RF transmitter 262 transmits the forehead location programming sequence 263 (in this example) through panel 230 to the subject wireless sensor 140. In response to this transmission, wireless sensor 140 (as previously programmed to sense temperature in FIG. 2B) processes the received forehead location programming sequence 263 which results in initialization (calibration, parameter setting, etc.) of now temperature sensor 140 for use on the patient's forehead. This is in accordance with the sequence of caregiver selections from sensor function panel 210 and body placement panel 230.

Actuators 261 may be pressure induced, heat activated, light sensitive, or of other activation technology. Like in FIG. 2B, the caregiver may press sensor 140 against the indicia of body placement panel 230 to generate corresponding actuators 261, or may otherwise operate actuators 261 of desired body placement indicia and then hold sensor 140 near or adjacent to the indicia for operation of the corresponding initialization circuit.

The activation and programming sequence for the body placement location may be, for example the case of placing the sensor on the chest of a patient to monitor respiration. In this case, “R” 220d (on sensor function panel 210) is depressed by the caregiver, before both 246L and 246R on body placement panel 230 are depressed by the caregiver. Depressing “R” 220d may activate in this case the sensor's strain gauge function, and the two location indicia 246L, 246R would indicate that the sensor is to be programmed for use/placement across the chest of the patient. In another example, a patient with a badly sprained right leg may be monitored for motion by the caregiver pressing sensor 140 on function indicia “M” 220b and then subsequently depressing Anterior indicia 234 and holding sensor 140 against upper left leg indicia 250L on body placement panel 230. This sequence programs the sensor 140 to monitor right leg motion for a patient who may be undergoing therapy, for example.

Numerical indicia 290 may also be included to program the sensor with patient information. The patient identification information can be a patient's social security number, hospital identification number, or any other unique numerical value. Behind each of numerical indicia 290 is an associated initialization circuit which electronically communicates the numerical number associated with the indicia to the wireless sensor 140 (FIG. 1) through the initialization unit 150.

Optional programming display lights 298a, 298b and a speaker 296 may be used either separately or collectively to aid the user in initializing or otherwise programming the wireless sensor 140 (FIG. 1). Further, an optional slot 294 may be provided for programming the wireless sensor 140 (FIG. 1) where the initialization circuitry is located within the slot for communicating to the sensor 140 information represented by indicia as selected by the user/care provider. Slot 294 may be used as an alternative to or in combination with the present holding of sensor 140 against panels 210, 230 described above.

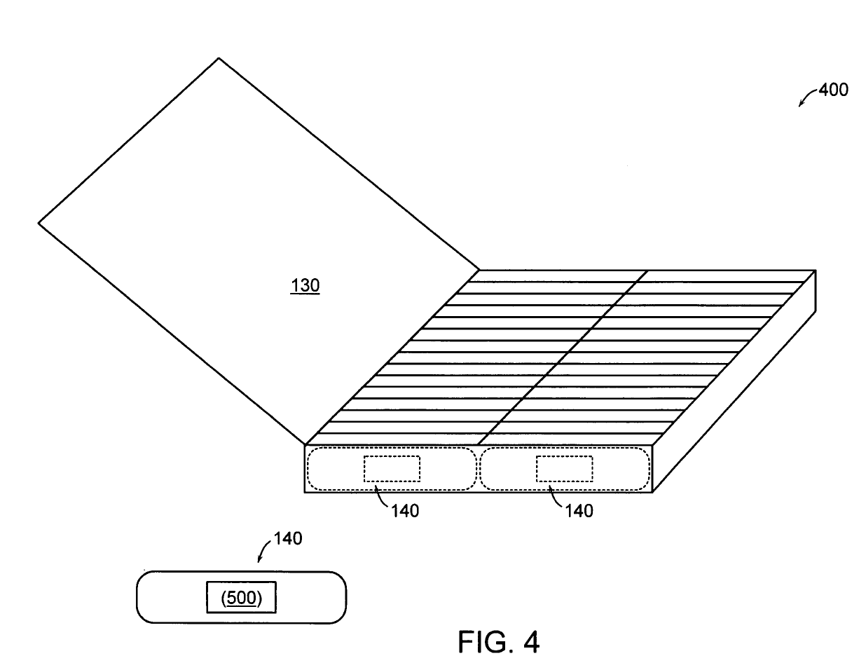

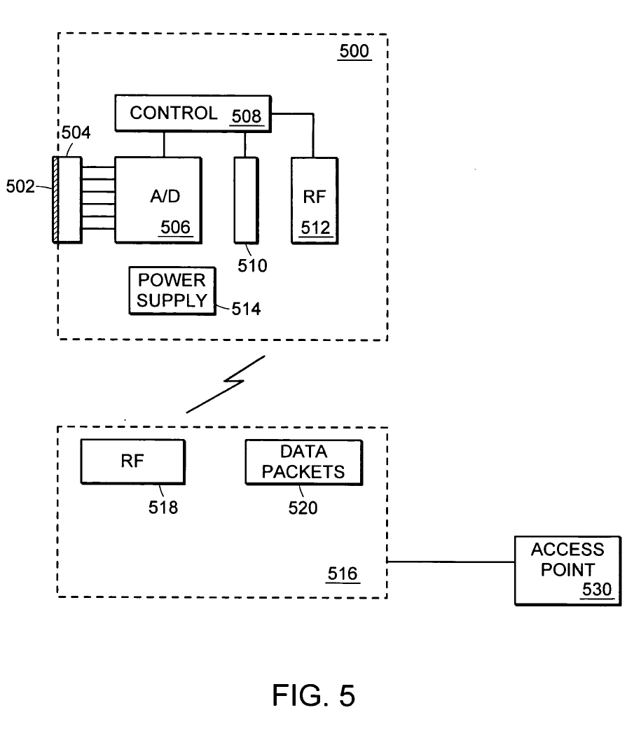

FIG. 4 shows a perspective view of a box 130 or kit 400 of generic wireless sensors 140. The sensor 140 has an adhesive backing strip or substrate (similar to that of a band-aid) and houses electronics 500 as shown in detail in FIG. 5. The electronics 500 include a power supply 514, a plurality of body contacts 502 for interfacing with the subject patient. Signals generated by the body contacts 502 are transmitted through a plurality of transducer elements 504 to an analog-to-digital (A/D) converter 506. The digitized output from A/D converter 506 is input to a programmable control unit 508 which is controlled by control logic 510. A radio frequency (RF) transceiver 512, transmits sensor output data to a base unit 160 (FIG. 1). The programmable control unit 508 is initialized (i.e., programmed) by sensor initialization device unit 150 (FIG. 2) to perform the user selected functions (e.g. sense temperature of the head, sense blood pressure from the hand, sense pulse or heart rate from the client area, etc.) as communicated: (a) from the user through the sensor function panel 210 and the body placement panel 230 (discussed above); and (b) from the initialization device 150 to the sensor electronics 500 via RF transceiver 512. That is, at the factory, the programmable control unit 508 is printed with all circuits for monitoring the full range of available functions (biological/physiological properties). At the time of use, through invention initialization unit 150, the programmable control unit 508 circuits corresponding to user selected/specified function(s) are initialized. After initialization/activation, the sensor electronics 500 serve the user selected functions only.

The control logic 510 supports operation of programmable control unit 508 and controls the flow of sensor data to and from the RF transceiver 512. Techniques common in the art for controlling data flow are employed.

Continuing with the lower portion of FIG. 5, the base unit 160 (FIG. 1) has similar electronics 516 for collecting and aggregating information (data) received from the wireless sensors 140 and sending the data to an access point 530. The base unit electronics 516 include an RF transceiver 518 and a data packetizer 520. The collecting and aggregating of data can be sent to the access point 530 over a 802.11or similar type protocol. Access point 530 may be a server in a network, a host computer, and the like, local and/or remote the patient location.

For example, a care giver can monitor, from a remote location, the sensed function for a given body placement location. Further, the care giver can remotely change the sensed function for the given body placement location by knowing the patient identification information and the body placement location. The caregiver communicates desired change in sensor function by transmitting a corresponding program sequence (similar to program sequences 223, 263 of FIGS. 2B and 2C) from the remote location to access point 530. In turn, access point 530 transmits the subject program sequence(s) to the base unit 160. In response to program sequences received at the base unit 160, RF transceiver 518 transmits the program sequences to sensor 140's RF transceiver 512. RF transceiver 512 processes the received program sequences similar to original initialization previously described. This results in changed or added sensor function of sensor 140 (and operates at the previously programmed body placement on the patient).

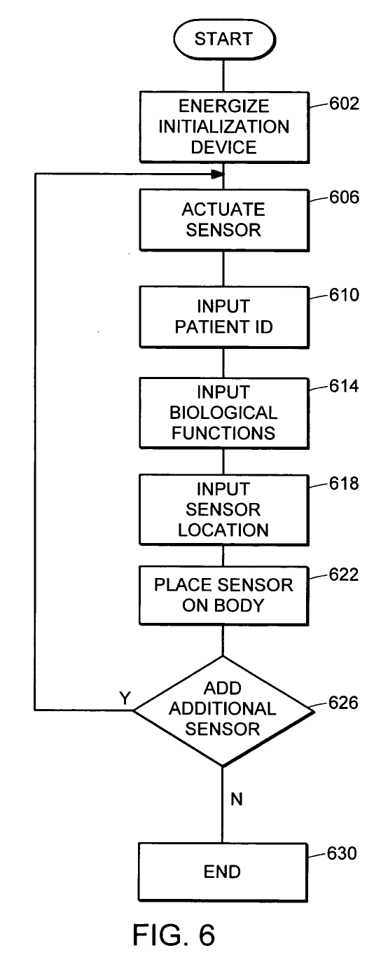

FIG. 6 is a flow diagram of sensor 140 initialization. The initialization of a wireless sensor 140 will be described using the flow diagram of FIG. 6 in conjunction with reference to FIGS. 1-5.

The initialization method starts with Step 602. In Step 602, the initialization unit 150 is energized (powered on). A care giver can simply energize the initialization panel by using power switch 202.

In Step 606, a wireless sensor 140 is activated. In one embodiment, the wireless sensor 140 is activated by removal of a non-conductive strip located between the power supply 514 and the power supply connector (to electronics 500).

In Step 610, patient identification information is input into activated sensor 140. The wireless sensor 140 is placed over and depressed on a numerical indicia 290 representing the first numeral of the patient's identification information. This step is repeated until the remaining numerals of the patient's identification information are programmed into the sensor 140. At each repetition an optional light 298 or audible signal (through speaker 296) can give an indication if the sensor 140 was programmed with or without error. Specifically, with each pressing of the sensor 140 against panel 230 indicia. Corresponding circuits coupled to the panel indicia transmit data signals to the sensor transceiver 512. Sensor control logic 510 and programmable control unit 508 coupled to transceiver 512 receive the data and store the patient's identification information. The patient identification information can be a patient's social security number, hospital identification number, or any other unique numerical value.

In Step 614, the user selected biological/physiological function is input (communicated) to the sensor 140. The wireless sensor 140 is placed over and depressed on biological/physiological function indicia 220a . . . 220n representing the user desired biological/physiological function to be sensed. Corresponding circuits coupled to panel 210 indicia transmit programming signals to the sensor transceiver 512. Sensor control logic 510 and programmable control unit 508 receive the programming signals from transceiver 512. In turn, the received programming signals initialize the sensor circuits that correspond to the user selected functions. An optional light 298 or audible signal (through speaker 296) can give an indication if the sensor 140 was programmed with or without error.

In Step 618, body placement information is input into the sensor 140. The wireless sensor 140 is placed over and depressed on body placement indicia 234, 236, 240, 260, 270, and 280 representing the area of the patient's body to be sensed. Corresponding panel 230 indicia circuits transmit data signals to the sensor transceiver 512. Sensor control logic 510 and programmable control unit 508 receive the data signals from transceiver 512 and determine therefrom body location where the sensor is to be used. In turn, sensor circuits 500 may calibrate and set certain variables accordingly. An optional light 298 or audible signal (through speaker 296) can give an indication if the sensor 140 was programmed with or without error. This step may be repeated until a precise location is programmed.

For example, a pulse located in the front of a patient's right foot is desired. The sensor 140 is placed and depressed on the following indicia in the following sequence: body function indicia “P” 220c, anterior indicia 234, lower right leg 252R, and foot 280. It should be understood that a variety of sequences can accomplish the same end result.

Continuing with FIG. 6, next (Step 622) the sensor 140 is removably affixed to the patient's body 110. A care giver places the sensor 140 on the patient's body 110 in the programmed location.

In Step 626, additional sensors 140 may be initialized/programmed by the user. If the desired sensed function requires additional sensors, such as for an electrocardiogram, steps 606 through 622 are repeated with another wireless sensor 140 from box 130 or kit 400.

The method ends at Step 630.

While this invention has been particularly shown and described with references to preferred embodiments thereof, it will be understood by those skilled in the art that various changes in form and details may be made therein without departing from the scope of the invention encompassed by the appended claims.

For example, human animal, or other subjects may benefit from the wireless sensors and systems of the present invention. As such, panel 230 indicia may be outlines and regions appropriate to such use. The illustrations in FIG. 2 are by way of example for human patients and are not limitations of the present invention. The panels may be replaced by a graphical user interface (GUI) for use with slot 294. The GUI may include a pressure or light sensitive touch screen similar to a PDA screen.

Alphabetic, other characters, and symbols common in the industry of use (medical, veterinarian, etc.) may be used in panel indicia 290 instead of or in addition to numerals. The numeric illustration and discussion in FIGS. 2 and 6 above are for purposes of illustrating, and not limiting, the principals of the present invention.

The initialization device 150 panel may employ a “smart” panel used in conjunction with a patient/hospital data base system for downloading to the panel patient information, such as gender, age, height, weight, etc. A “smart” panel may also include a bar code reader which may allow the caregiver to scan the patient's hospital wrist band for direct input of patient information to the sensor 140 rather than through the keypad 290.

1. A sensor initialization device, comprising:
a panel having respective indicia for each of plural wireless sensor functions, such that there are a plurality of sensor function indicia; and
for each sensor function indicia, a respective initialization circuit coupled to the sensor function indicia for electronic communication through the panel to a wireless sensor unit upon selection of the sensor function.

2. The sensor initialization device of claim 1, wherein the sensor function include body temperature, heart rate, blood pressure, respiratory, audio, electrocardiogram, electroencephalogram, electromyography, electrooculogram, and polysomnography.

3. The sensor initialization device of claim 1, further including:
the panel having respective indicia for each of plural body placement locations, such that there are a plurality of body placement location indicia; and
for each body placement location indicia, a respective initialization circuit coupled to the body placement location indicia for electronic communication through the panel to the wireless sensor unit upon selection of the body placement location indicia.

4. The sensor initialization device of claim 3, wherein the body placement location indicia include at least one of a pictorial image of body parts and a pictorial outline of a body with plural user selectable portions.

5. The sensor initialization device of claim 3, wherein the pictorial outline of a body is segmented into quadrants defining specific regions of the body.

6. The sensor initialization device of claim 3, wherein the pictorial outline of a body includes a detailed anterior outline of the body and a detailed posterior outline of the body.

7. The sensor initialization device of claim 3, further including an anterior indicia and a posterior indicia for selecting between a side of a subject body.

8. The sensor initialization device of claim 3, further including patient identification indicia for programming the sensor with patient identification information.

9. The sensor initialization device of claim 8, wherein the patient identification indicia are numerical indicia.

10. A generic biosensor, comprising:
a plurality of configurable sensors for sensing biological or physiological functions;
a control unit for programming the plurality of sensors to sense at least one respective biological or physiological function; and
a transceiver for transmitting the at least one sensed biological or physiological function to a base unit and for receiving programming information from an initialization unit.

11. The biosensor of claim 10, wherein the configurable sensors include a temperature sensor, a heart rate sensor, a blood pressure sensor, a respiratory sensor, an audio sensor, an electrocardiogram sensor, an electroencephalogram sensor, an electromyography sensor, an electrooculogram sensor, and a polysomnography sensor.

12. The biosensor of claim 10, further including unique sensor identification information.

13. The biosensor of claim 12, wherein the sensor identification information is a sensor serial number.

14. The biosensor of claim 10, wherein the control unit further programs the biosensor with patient identification information.

15. A method of initializing a generic biosensor; comprising:
activating the biosensor;
programming the biosensor with patient identification information;
determining at least one biological or physiological function to be sensed;
programming the biosensor for the determined biological or physiological function using an indicia representing the determined biological or physiological function;
determining a body location where the programmed bio-sensor is to be placed on a subject; and
programming the biosensor for the determined body location using an indicia representing the determined body location.

16. The method of claim 15, wherein the patient identification information includes a patient's social security number or a patients hospital identification number.

17. The method of claim 15, wherein programming the biosensor with patient identification information includes depressing the biosensor on a panel bearing indicia representing the patient identification information.

18. The method of claim 17, wherein the indicia are numeral indicia.

19. The method of claim 15, wherein programming the biosensor for the determined biological or physiological function includes depressing the biosensor on a panel bearing biological or physiological function indicia representing the determined biological or physiological function.

20. The method of claim 19, wherein the biological or physiological function include body temperature, heart rate, blood pressure, audio, respiratory, electrocardiogram, electroencephalogram, electromyography, electrooculogram, and polysomnography.

21. The method of claim 20, wherein programming the biosensor for the determined body location includes depressing the bio-sensor on a panel bearing body location indicia representing the determined body location.

22. The method of claim 21, wherein the step of depressing the biosensor on a panel further includes depressing the biosensor on multiple body location indicia on the panel until the biosensor is fully programmed with the determined body location.

23. The method claim 21, wherein the body location indicia include at least one of a pictorial image of body parts and a pictorial outline of a body with plural user selectable portions.

24. The method of claim 23, wherein the pictorial outline of a body is segmented into quadrants defining specific regions of the body.

25. The method of claim 23, wherein the pictorial outline of a body includes a detailed anterior outline of the body and a detailed posterior outline of the body.
